(19) United States
(12) Patent Application Publication
(10) Pub. No.: US 2026/0196245 A1
(43) Pub. Date: Jul. 9, 2026

(54) METHODS AND SYSTEMS FOR RECORDING ULTRASOUND VIDEOS FOR AUTOMATED ANALYSIS BY ARTIFICIAL INTELLIGENCE/DEEP LEARNING (AI/DL) ALGORITHMS

(71) Applicant: MIM Fertility Sp. z. o.o., Warszawa (PL)

(72) Inventor: Piotr Wygocki, Warszawa (PL)

(73) Assignee: MIM Fertility Sp. z. o.o., Warszawa (PL)

(21) Appl. No.: 19/090,980

(22) Filed: Mar. 26, 2025

Related U.S. Application Data
(60) Provisional application No. 63/741,692, filed on Jan. 3, 2025.

(51) Int. Cl. G11B 20/10; A61B 8/00; A61B 8/08

(52) U.S. Cl. CPC G11B 20/10527; A61B 8/0858; A61B 8/42; A61B 8/461; A61B 8/5223

(57) ABSTRACT

Disclosed herein are computer-implemented systems and methods for recording ultrasound videos, specifically designed to facilitate automated analysis by artificial intelligence (AI) and deep learning (DL) algorithms. The method involves capturing a cine-loop (a continuous video segment) in a standardized manner to enhance the accuracy and efficiency of AI/DL algorithms in medical imaging and diagnostics. The method includes selecting an appropriate preset on the ultrasound device for a target organ, positioning a probe to center the target organ on a screen, adjusting scanning depth, enabling harmonic or spectral imaging, and scanning the target organ while recording the cine-loop with a steady, uniform motion, maintaining a constant scanning plane. The recording is saved and reviewed for quality, allowing for parameter adjustments in future scans. The method of the present disclosure improves the automated interpretation of ultrasound videos, particularly in gynecological studies, including in vitro fertilization (IVF).

### CROSS-REFERENCE TO RELATED APPLICATIONS

This application claims benefit of priority to U.S. Provisional Patent Application No.: 63/741,692, filed on Jan. 3, 2025, titled “METHODS AND SYSTEMS FOR RECORDING ULTRASOUND VIDEOS FOR AUTOMATED ANALYSIS BY ARTIFICIAL INTELLIGENCE/DEEP LEARNING (AI/DL) ALGORITHMS, TRIGGER DAY PREDICTION, AND PROVIDING RECOMMENDATION FOR IN VITRO FERTILIZATION”, the entirety of which is incorporated herein by reference.

### FIELD OF TECHNOLOGY

Aspects of the present disclosure relate to technology in medical imaging. In particular, the present disclosure encompasses the intersection of medical imaging and artificial intelligence. In one aspect, the present disclosure addresses technologies at the intersection of ultrasound and artificial intelligence, with a focus on improving the acquisition of video data to facilitate the development and application of Artificial Intelligence/Deep Learning (AI/DL) algorithms in gynecology and reproductive health.

The method of the present disclosure aims to bridge the gap between the capabilities of modern ultrasound devices and the requirements of cutting-edge AI/DL techniques, ultimately leading to more accurate and efficient diagnostic tools in these medical domains.

### BACKGROUND

Ultrasound imaging has long been a cornerstone of medical diagnostics, particularly in the fields of gynecology and reproductive health. Traditional ultrasound techniques rely heavily on the skill and experience of the sonographer to capture and interpret images. While these methods have proven valuable, they are subject to human variability and limitations in consistency and reproducibility.

In recent years, there has been a growing interest in applying Artificial Intelligence (AI) and Deep Learning (DL) algorithms to medical imaging analysis. These technologies have shown great promise in enhancing diagnostic accuracy, reducing interpretation time, and potentially identifying subtle patterns that might be overlooked by human observers. However, the effectiveness of AI/DL algorithms in ultrasound analysis is heavily dependent on the quality and consistency of the input data.

Existing methods for recording ultrasound videos often lack standardization, leading to variability in image quality and content. This variability can significantly impact the performance of AI/DL algorithms, as these systems require consistent, high-quality data for optimal training and operation. Current practices may include one or more of the following:
1) Freehand scanning: Sonographers move the ultrasound probe freely, which can result in inconsistent coverage of the target organ and variable image quality.
2) Static image capture: Many practitioners focus on capturing still images rather than video sequences, limiting the temporal information available for analysis.
3) Non-standardized settings: Ultrasound machine settings, such as depth, gain, and imaging mode, are often adjusted based on individual preference rather than standardized protocols.
4) Inconsistent sweep techniques: When video sequences are captured, the speed and steadiness of the probe movement can vary significantly between operators and examinations.

The inconsistencies in data acquisition pose significant challenges for the development and application of AI/DL algorithms in ultrasound analysis. The inconsistencies can lead to reduced accuracy, increased false positives or negatives, and limited generalizability of AI models across different clinical settings. Furthermore, existing techniques primarily focus on processing and analysis of ultrasound images rather than the standardization of the image acquisition process itself. In addition, improving the image acquisition process is critical for diagnostic tools such as Assisted Reproductive Technologies (ARTs), which are used in reproductive medicine, more specifically, during in vitro fertilization (IVF).

Therefore, there is a need for a standardized method of recording ultrasound videos that is specifically optimized for AI/DL analysis. Furthermore, there is a need to address the current limitations in data consistency and quality, while also being practical for implementation in clinical settings.

### SUMMARY

Aspects of the disclosure relate to a novel method for recording ultrasound videos specifically designed to facilitate automated analysis by artificial intelligence (AI) and deep learning (DL) algorithms. This method addresses the critical need for standardized, high-quality ultrasound data, particularly in the fields of gynecology and reproductive health, enabling more accurate and efficient AI-driven diagnostics. The present disclosure aims to close the gaps described above by providing a structured, reproducible approach to ultrasound video capture that enhances the capabilities of AI/DL algorithms in gynecological and reproductive health applications.

In particular, the method of the present disclosure comprises a series of carefully defined steps for capturing a cine-loop (a continuous video segment) of a target organ. These steps are optimized to produce ultrasound videos that are ideally suited for subsequent AI/DL analysis. The key components of the method of the present disclosure include:
1) Standardized preset selection and probe positioning;
2) Precise adjustment of scanning depth and image enhancement settings;
3) Systematic selection of scanning plane and initial positioning;
4) Controlled cine-loop recording with a uniform sweeping from one end of the target organ to an opposite end of the target organ; and
5) Standardized saving and review process.

The method of the present disclosure offers several significant advantages over existing ultrasound recording techniques, includes at least the following:
1) Consistency: By following a standardized procedure, the method reduces variability between different operators and examinations, providing more consistent input for AI/DL algorithms;
2) Optimization for AI/DL: Each step is specifically designed to enhance features that are crucial for AI/DL analysis, such as complete organ coverage and uniform scanning speed;
3) Improved image quality: The method incorporates steps to optimize image clarity and detail, which are essential for accurate AI/DL interpretation.
4) Efficiency: The streamlined process can be quickly learned and implemented in clinical settings without significant disruption to existing workflows.
5) Versatility: While primarily focused on gynecological applications, the method can be adapted for use with various ultrasound machines and potentially extended to other medical fields.

The method of the present disclosure also encompasses a system and a computer program product that implement this method, allowing for seamless integration into existing ultrasound equipment and clinical processes.

Thus, by providing a standardized approach to ultrasound video capture, the method of the present disclosure has the potential to significantly enhance the accuracy and reliability of AI/DL algorithms in medical imaging analysis. This, in turn, could lead to improved diagnostic capabilities, more personalized treatment plans, and ultimately better patient outcomes in gynecology and reproductive health.

According to one example aspect of the disclosure, a computer-implemented method for recording ultrasound videos to facilitate automated analysis by AI and DL algorithms is provided, the method comprising: selecting an appropriate preset on an ultrasound device for an application at least in part based on a target organ; positioning a probe of the ultrasound machine thereby positioning an image of the target organ at a center of a screen of a display device; performing adjustments of scanning depths until an entirety of the target organ is visualized with minimal surrounding structures; performing image enhancements, the performing of the image enhancements including one or more of: enabling Harmonic Imaging (HI) or Full Spectral Imaging (FSI), when the HI or FSI enhancement modes are available on the ultrasound machine, adjusting gains and/or contrasts when adjustments to gains or contrasts are needed; selecting a scanning plane for a complete sweep from one end of the target organ to an opposite end of the target organ; performing an initial positioning of the probe; initiating the cine-loop recording; scanning the target organ from the one end of the target organ to the opposite end of the target organ while recording the cine-loop, the scanning being performed with a steady, uniform motion, maintaining a constant scanning plane; and ending the cine-loop recording when the scanning is completed, and storing the recorded cine-loop in a memory of the ultrasound machine.

In one example aspect, the computer-implemented method further comprises: performing a review of the recorded cine-loop for quality, and determining whether the recorded cine-loop satisfied criteria for subsequent analysis.

In one example aspect, the computer-implemented method further comprises: providing an operator of the ultrasound machine an opportunity to adapt techniques used during the scanning for future scans

In one example aspect, the target organ is an ovary or a uterus and the method is used to conduct gynecological studies, the gynecological studies comprising at least simulations of in vitro fertilization (IVF).

In one example aspect, the target organ is the ovary and the method is used for counting and measuring follicles in the gynecological studies, the gynecological studies comprising at least the simulations of the IVF.

In one example aspect, the target organ is the uterus and the method is used for measuring an endometrium thickness in the gynecological studies, the gynecological studies comprising at least the simulations of the IVF.

In one example aspect the cine-loop recording is optimized for use with a specific ultrasound machine, the specific ultrasound machine being at least an ultrasound machine selected from: a GE Voluson ultrasound machine, a Siemens ultrasound machine, a Samsung ultrasound machine, a Philips ultrasound machine, a Canon Aplio ultrasound machine, a Mindray ultrasound machine, a Fujifilm ultrasound machine, a BK Medical ultrasound machine, and a Hitachi ultrasound machine.

According to one example aspect of the disclosure, a computer-implemented system is provided for recording ultrasound videos to facilitate automated analysis by AI and DL algorithms, the system comprising: at least one memory; and at least one hardware processor coupled with the at least one memory and configured, individually or in combination, to: select an appropriate preset on an ultrasound device for an application at least in part based on a target organ; position a probe of the ultrasound machine thereby positioning an image of the target organ at a center of a screen of a display device; perform adjustments of scanning depths until an entirety of the target organ is visualized with minimal surrounding structures; perform image enhancements, the performing of the image enhancements including one or more of: enable Harmonic Imaging (HI) or Full Spectral Imaging (FSI), when the HI or FSI enhancement modes are available on the ultrasound machine, adjusting gains and/or contrasts when adjustments to gains or contrasts are needed; select a scanning plane for a complete sweep from one end of the target organ to an opposite end of the target organ; perform an initial positioning of the probe; initiate the cine-loop recording; scan the target organ from the one end of the target organ to the opposite end of the target organ while recording the cine-loop, the scanning being performed with a steady, uniform motion, maintaining a constant scanning plane; and end the cine-loop recording when the scanning is completed, and store the recorded cine-loop in a memory of the ultrasound machine.

According to one example aspect of the disclosure, a system for recording ultrasound videos of a target organ is provided, the system comprising: a computing with memory, the computer including at least one processor; a display device communicatively coupled to the computing device; a beamforming equipment communicatively coupled to the display device and the computer; an ultrasound machine for performing scans comprising a transducer coupled to the beamforming equipment; and a navigation support program stored on the memory of the computer, the navigation support program including software code, the code when executed by the processor of the computer performing: capturing of images of an organ within a body using the ultrasound machine; and processing information gathered during the capturing of the images to create a repeating video sequence optimized for automated analysis by an AI and/or DL based algorithm.

According to one example aspect of the disclosure, a computer program product for ultrasound navigation during recording of videos of a target organ is described, the computer program product comprising instructions stored on a computer-readable medium, when executing by a computing device, the instructions enabling the computer to perform: receiving ultrasound data of the target organ in raw form an ultrasound machine when the ultrasound machine performing scanning; processing the received ultrasound data to generate a cine-loop optimized for automated analysis by an AI and/or DL based algorithm in accordance with the aspects of the present disclosure including: selecting an appropriate preset on an ultrasound device for an application at least in part based on a target organ; positioning a probe of the ultrasound machine thereby positioning an image of the target organ at a center of a screen of a display device; performing adjustments of scanning depths until an entirety of the target organ is visualized with minimal surrounding structures; performing image enhancements, the performing of the image enhancements including one or more of: enabling Harmonic Imaging (HI) or Full Spectral Imaging (FSI), when the HI or FSI enhancement modes are available on the ultrasound machine, adjusting gains and/or contrasts when adjustments to gains or contrasts are needed; selecting a scanning plane for a complete sweep from one end of the target organ to an opposite end of the target organ; performing an initial positioning of the probe; initiating the cine-loop recording; scanning the target organ from the one end of the target organ to the opposite end of the target organ while recording the cine-loop, the scanning being performed with a steady, uniform motion, maintaining a constant scanning plane; ending the cine-loop recording when the scanning is completed; and storing the generated cine-loop in a memory of the computing device.

According to one example aspect of the disclosure, a non-transitory computer-readable medium is provided storing a set of instructions thereon for recording ultrasound videos to facilitate automated analysis by AI and DL algorithms, wherein the set of instructions comprises instructions for: selecting an appropriate preset on an ultrasound device for an application at least in part based on a target organ; positioning a probe of the ultrasound machine thereby positioning an image of the target organ at a center of a screen of a display device; performing adjustments of scanning depths until an entirety of the target organ is visualized with minimal surrounding structures; performing image enhancements, the performing of the image enhancements including one or more of: enabling Harmonic Imaging (HI) or Full Spectral Imaging (FSI), when the HI or FSI enhancement modes are available on the ultrasound machine, adjusting gains and/or contrasts when adjustments to gains or contrasts are needed; selecting a scanning plane for a complete sweep from one end of the target organ to an opposite end of the target organ; performing an initial positioning of the probe; initiating the cine-loop recording; scanning the target organ from the one end of the target organ to the opposite end of the target organ while recording the cine-loop, the scanning being performed with a steady, uniform motion, maintaining a constant scanning plane; and ending the cine-loop recording when the scanning is completed, and storing the recorded cine-loop in a memory of the ultrasound machine.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations. The foregoing and other objects, features, and advantages will be apparent from the following more detailed description of exemplary aspects, as illustrated in the accompanying drawings. In the drawings, like reference characters generally refer to the same parts throughout the different views. The drawings are provided for illustrative purposes and are not necessarily to scale, with emphasis instead placed on illustrating the principles of the various aspects.

The accompanying drawings, which are incorporated into and constitute a part of this specification, illustrate one or more example aspects of the present disclosure and, together with the detailed description, serve to explain their principles and implementations. The foregoing and other objects, features, and advantages will be apparent from the following more detailed description of exemplary aspects, as illustrated in the accompanying drawings. In the drawings, like reference characters generally refer to the same parts throughout the different views. The drawings are provided for illustrative purposes and are not necessarily to scale, with emphasis instead placed on illustrating the principles of the various aspects.

FIG. 1 illustrates a high-level block diagram of a system for recording ultrasound videos to facilitate automated analysis by AI and DL algorithms in accordance with aspects of the present disclosure.

FIG. 2 presents a flowchart of a method for recording ultrasound videos optimized for automated analysis by Artificial Intelligence or Deep Learning based algorithms in accordance with aspects of the present disclosure.

FIG. 3 illustrates a block diagram of an exemplary user interface on an ultrasound machine with locations of key controls in accordance with aspects of the present disclosure.

FIG. 4 illustrates a probe positioning diagram showing a correct probe positioning for a uterus in accordance with aspects of the present disclosure.

FIG. 5 depicts different scanning planes and a process of selecting an optimal scanning place for an ovary in accordance with aspects of the present disclosure.

FIG. 6 illustrates a block diagram showing a sweeping motion of a probe during cine-loop recording in accordance with aspects of the present disclosure.

FIG. 7 depicts images illustrating effects of adjustments of scanning depths on ultrasound images in accordance with aspects of the present disclosure.

FIG. 8 depicts images illustrating effects of harmonic or spectral image enhancements in accordance with aspects of the present disclosure.

FIG. 9 shows a screenshot displaying a cine-loop during a review process on an ultrasound machine in accordance with aspects of the present disclosure.

FIG. 10 illustrates a high-level block diagram showing the feeding of recorded cine-loops into an AI/DL algorithm based analyzer in accordance with aspects of the present disclosure.

FIG. 11 illustrates a side-by-side comparison of traditional ultrasound recording techniques with the ultrasound recording method in accordance with aspects of the present disclosure.

FIG. 12 illustrates high-level block diagrams of exemplary ultrasound machines on which the method of present disclosure is implemented in accordance with aspects of the present disclosure.

FIG. 13 is a block diagram illustrating various components of an example computer system via which aspects of the present disclosure may be implemented.

FIG. 14 is a block diagram of various example system components, usable in accordance with aspects of the present disclosure.

### DETAILED DESCRIPTION

Example aspects are described herein in the context of an apparatus, system, method, and various computer program features for recording ultrasound videos to facilitate automated analysis by AI and DL algorithms. The method of the present disclosure is a comprehensive method and is specifically optimized for automated analysis by AI and DL algorithms. In one aspect, the method is particularly tailored for applications in gynecology and reproductive health. However, the principles of the present method may be extended to other medical fields.

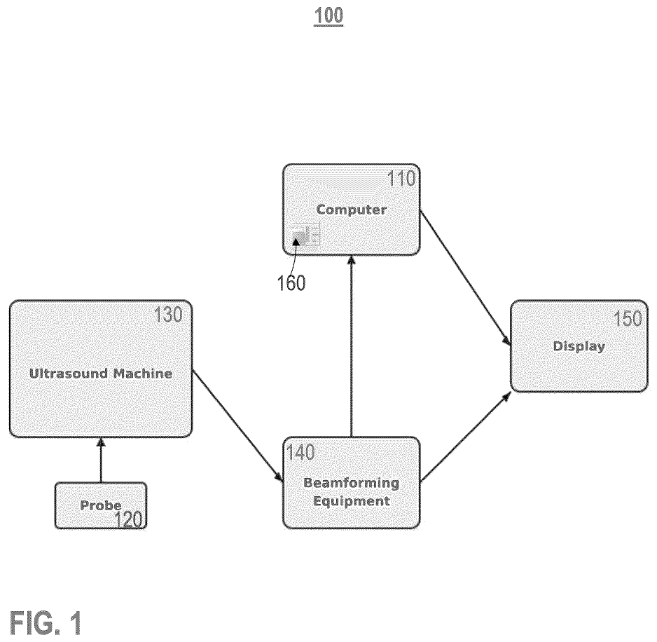

FIG. 1 illustrates a high-level block diagram of a system 100 for recording ultrasound videos to facilitate automated analysis by AI and DL algorithms in accordance with aspects of the present disclosure. The system 100 includes a computer 110 with memory and at least one processor, a probe 120, an ultrasound machine 130 with a transducer linked to a beamforming equipment 140, the beamforming equipment 140, and a display device 150, and a navigation support program 160 running the memory of the computer 110. The probe 120 is communicatively coupled to the ultrasound machine 130 and the beamforming equipment is communicatively coupled to the ultrasound machine 130, computer 110, and the display device 150. The display device 150 receives images and data to display from input from the computer 110 and the beamforming equipment 140, as needed. The probe 120 is positioned on the body of the patient, e.g., by a sonographer, such that the recording of images is performed for the appropriate body part (e.g., uterus) of the patient. Ultrasound images recorded by the ultrasound machine 130 are fed to the beamforming equipment 140. Once the recorded ultrasound images are processed by the beamforming equipment 140, the output is fed to the computer 110 and is displayed via the display device 150.

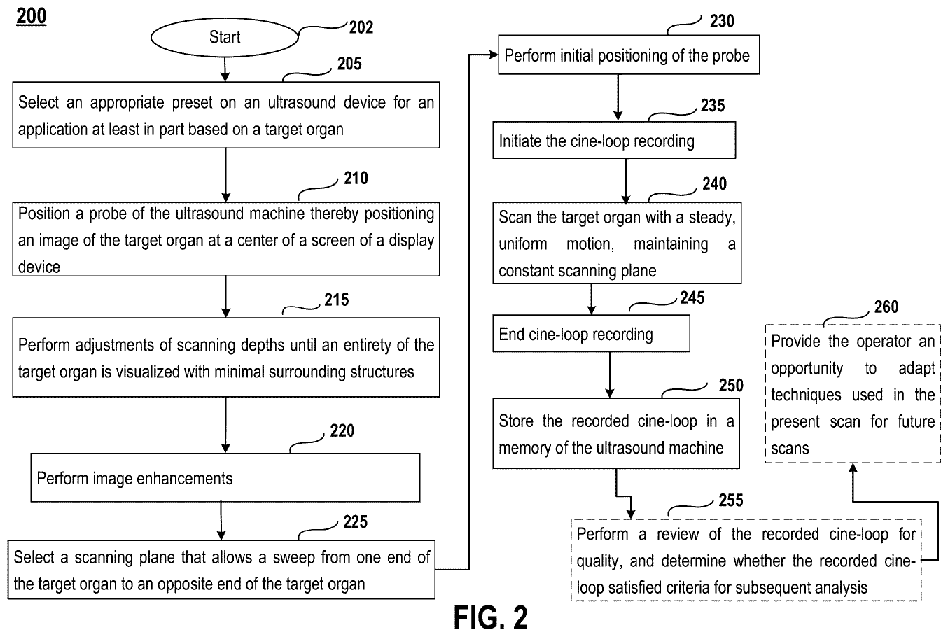

FIG. 2 presents a flowchart that illustrates a method 200 for recording ultrasound videos optimized for automated analysis by Artificial Intelligence or Deep Learning based algorithms in accordance with aspects of the present disclosure. Although method 200 may be implemented for recording ultrasound images in any medical field, the method is particularly exemplified for applications in gynecology and reproductive health. The following detailed description outlines each step of method 200, along with its rationale and potential variations in implementation of the particular step without further limiting the method of the present disclosure.

Method 200 starts in step 202 and proceeds to step 205.

In step 205, method 200 selects an appropriate preset on an ultrasound device for an application at least in part based on a target organ. For instance, for gynecological applications, this typically involves choosing presets such as “Follicle,” “Ovary,” “Gyne,” or “Adnexa.” The selection of the correct preset ensures that the initial settings of the ultrasound machine are optimized for the target organ, providing a consistent starting point for all examinations.

In step 210, method 200 positions a probe of the ultrasound machine thereby positioning an image of the target organ at a center of a screen of a display device, e.g., a screen of a display device 150. This step ensures that images of the organ of interest are fully captured in subsequent recordings of the cine-loop. Proper centering also aids the AI/DL algorithms in identifying relevant anatomical structures and in focusing on the most relevant anatomical structures.

In step 215, method 200 performs adjustments of scanning depths until an entirety of the target organ (i.e., the whole organ) is visualized with minimal surrounding structures.

In one aspect, step 215 is used for the following two reasons:
a) The adjustment of the scanning depth is used to ensure that the entire target organ is captured, providing complete information for analysis using the AI/DL based algorithm.
b) The adjustment of the scanning depth is used to minimize inclusion of irrelevant structures—thereby reducing noise and potential confounding factors during the subsequent analysis to be performed using the AI/DL based algorithm.

In one aspect, the optimal scanning depth is based on at least one of: the target organ, anatomy of a patient.

In one aspect, a sonographer or an operator of the ultrasound machine aims to have the image of the organ occupy approximately ⅔ of the depth of the screen for an ideal visualization of the target organ.

In step 220, method 200 performs image enhancements. In one aspect, the image enhancement includes enabling Harmonic Imaging (HI), or Full Spectral Imaging (FSI), when these enhancement modes are available on the ultrasound machine. The HI and FSI advanced imaging modes can significantly improve image quality by reducing artifacts and enhancing tissue differentiation. Then, in one aspect, the gain and contrast are adjusted to make details crisp and clear. However, it is important to note that no special tuning beyond standard optimization is necessary, as the AI/DL algorithms are designed to work with a range of image qualities.

In step 225, method 200 selects a scanning plane that allows a comfortable sweep from one end of the target organ to an opposite end of the target organ. This step is used for ensuring a smooth, consistent motion during the subsequent cine-loop recording.

In one aspect, the choice of scanning plane is based on the target organ and the specific diagnostic goals. For example, in ovarian examinations, a longitudinal plane is often preferred for follicle counting.

In step 230, method 200 performs an initial positioning of the probe. The probe is moved to one end of the target organ, starting on a frame where the target organ is not yet visible. This ensures that the boundary of the organ is completely recorded, providing crucial information for AI/DL based algorithms about the extent of the organ and the structures surrounding the target organ.

In step 235, method 200 initiates the cine-loop recording. In one aspect, the cine-loop recording is initiated by pressing a button, typically based on the specific ultrasound machine. For instance, the cine-loop recording may be initiated by pressing a “Freeze” button twice. The initiation of the cine-loop recording begins the capturing of a continuous video segment for subsequent analysis based on the AI/DL based algorithms.

In step 240, method 200 scans the target organ with a steady, uniform motion, maintaining a constant scanning plane.

In one aspect, a sweep of the scan takes approximately two seconds from one end of the target organ to the other end of the target organ (opposite end). The consistency of this motion is crucial for several reasons:
a) The consistency of the motion provides a uniform sampling of the entire target organ.
b) The consistency of the motion allows AI/DL algorithms to more easily track structures across frames.
c) The consistency of the motion reduces motion artifacts that could interfere with subsequent image analysis.

In step 245, method 200 ends the cine-loop recording. The cine-loop recording is ended as soon as the other end of the target organ is reached, or just a frame after. This is typically done by pressing the “Freeze” button again. Ending the recording promptly ensures that only relevant information is included in the cine-loop, thereby optimizing the data for subsequent analysis using the AI/DL based algorithm.

In step 250, method 200 stores the recorded cine-loop in a memory of the ultrasound machine. The storing of the recorded cine-loop is initiated in different ways based on the particular ultrasound machine. In one aspect, a specific button is pressed to save the recorded content into the memory. For example, for GE Voluson ultrasound machines, an operator presses a button “P2”,for Mindray ultrasound machines the operator presses a button “Save2”, and for Samsung ultrasound machines, the operator presses a button “Store/Save U4”. The saved recording of the cine-loop should appear on a list of saved images on the screen of the display device, thereby confirming successful storage into the memory of the ultrasound machine.

In step 255, method 200 performs a review of the recorded cine-loop for quality, and determines whether the recorded cine-loop satisfied criteria for subsequent analysis. In one aspect, the review is performed using a trackball. The review serves at least two purposes:
a) The review allows the operator to check if a good image was obtained, ensuring the quality of the data for AI/DL based analysis.
b) The review provides an opportunity for the operator to determine whether to adapt technique applied during the current scan for future scans, considering parameters such as scanning speed, depth, probe pressure, and angle. When the recorded cine-loop meets the criteria for subsequent analysis, the method proceeds to step 260.

In step 260, method 200 provides the operator an opportunity to adapt techniques used in the present scan for future scans. The adaptation of the techniques may include adjusting parameters for future scanning. In one aspect, the parameters adjusted for future scanning may be parameters associated with one or more of: scanning speed, scanning depth, pressure associated with placement of the probe on the body of the patient, angle of the probe during scanning, and so on.

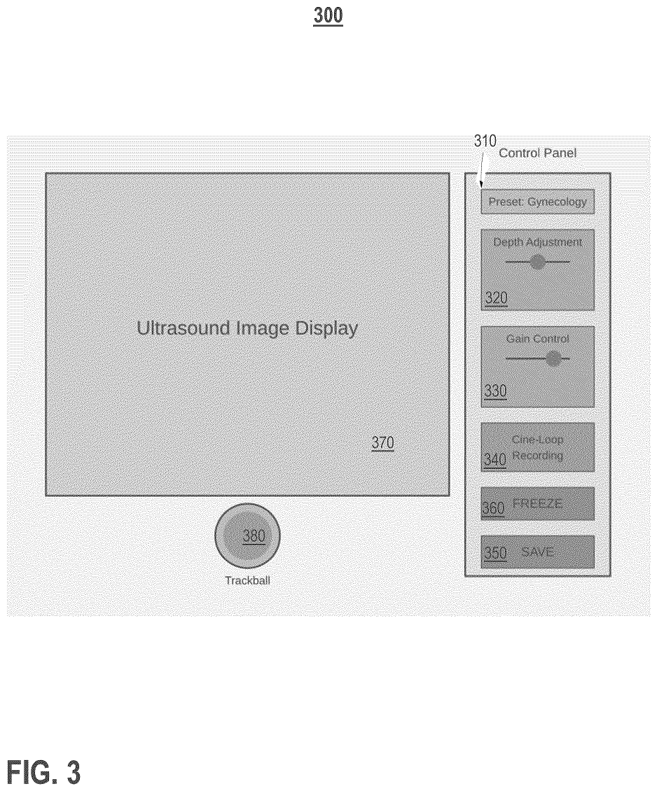

FIG. 3 illustrates a block diagram of an exemplary user interface 300 on an ultrasound machine with locations of key controls in accordance with aspects of the present disclosure. In one aspect, the key controls may include preset selections, depth adjustments, and cine-loop recording. The user interface 300 provides control panel with locations for key controls such as for: a key control 310 for selecting a preset for an application at least in part based on a target organ, a key control 320 for adjustments of scanning depths, a key control 330 for image enhancements, such as to adjust gain level, a key control 340 for initiates the cine-loop recording, a key control 350 for initiating the saving of the recorded cine-loop, and a key control 360 for showing recorded cite-loops. In addition, the user interface 300 displays the images captured via a display portion 370 of the user interface. In addition, the operator of the ultrasound machine may use trackball 380 to review the quality of the recorded images.

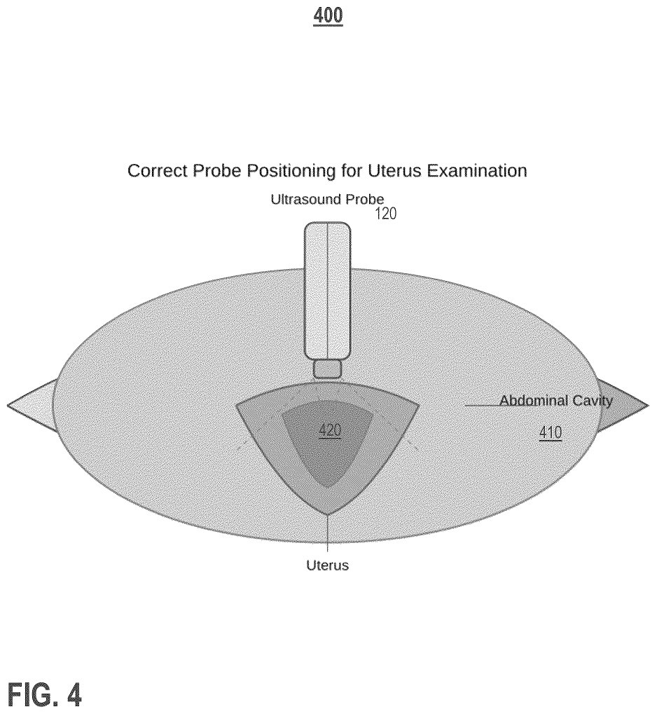

FIG. 4 illustrates a probe positioning diagram 400 showing a correct probe positioning for a uterus in accordance with aspects of the present disclosure. In one aspect, an ultrasound probe, such as the probe 120 of FIG. 1, is placed above the abdominal cavity 410 of the patient. The placement of the probe 120 allows the captured image to include relevant portions of the target organ, such as the uterus 420.

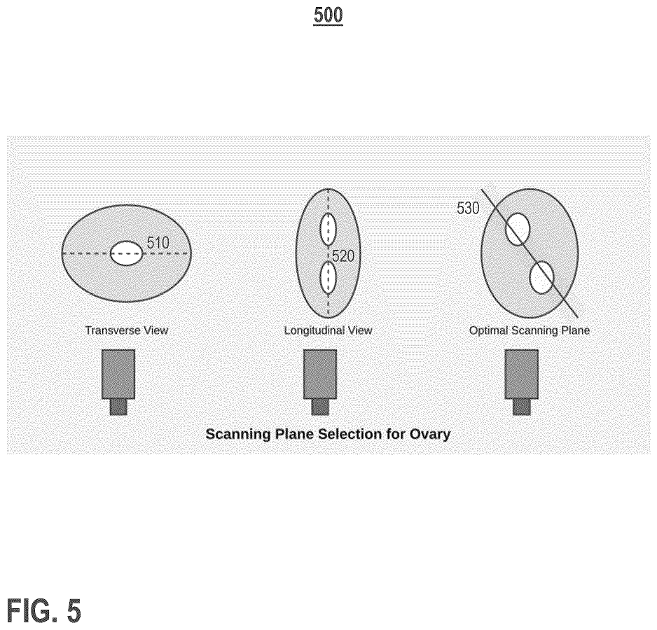

FIG. 5 depicts different scanning planes 500 and a process of selecting an optimal scanning plane for a target organ, e.g., an ovary, in accordance with aspects of the present disclosure. For example, a scanning plane 510 provides a transverse view of the target organ, scanning plane 520 shows a longitudinal view of the target organ, and scanning plane 530 shows an optimal scanning plane of the target organ. For the example ovary, the optimal scanning plane 530 is selected.

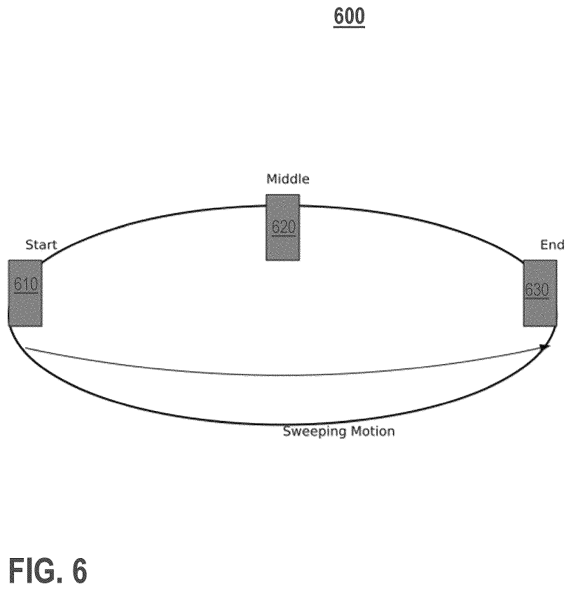

FIG. 6 illustrates a block diagram 600 showing a sweeping motion of a probe during cine-loop recording in accordance with aspects of the present disclosure. The sweeping motion is from a starting location 610 to an end location 630, through a middle location 620. The sweeping motion smooth and during the subsequent cine-loop recording.

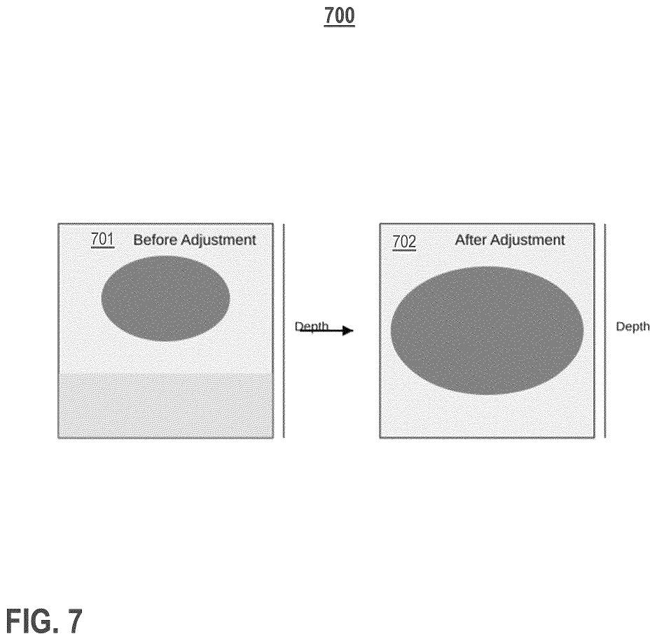

FIG. 7 depicts images 700 illustrating effects of adjustments of scanning depths on ultrasound images in accordance with aspects of the present disclosure. The image 701 illustrates an ultrasound image before an adjustment of a scanning depth. The image 702 illustrates the image after the scanning depth is adjusted.

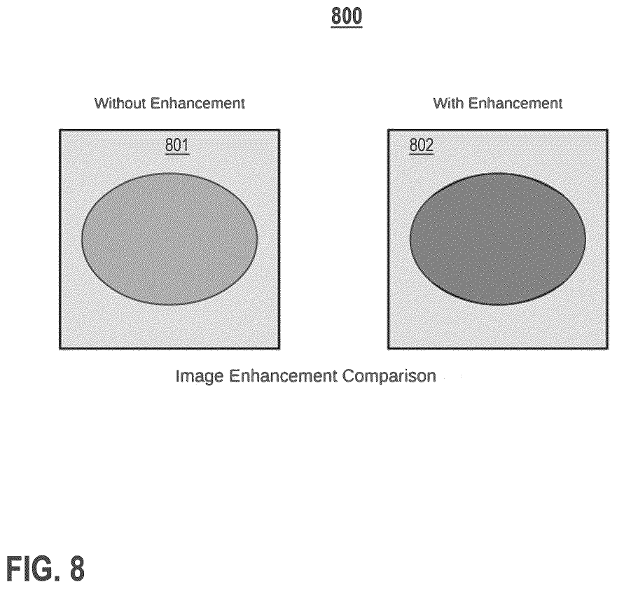

FIG. 8 depicts images 800 illustrating effects of harmonic or spectral image enhancements in accordance with aspects of the present disclosure. The image 801 illustrates an ultrasound image before harmonic or spectral image enhancements. The image 802 illustrates the ultrasound image after harmonic or spectral image enhancements.

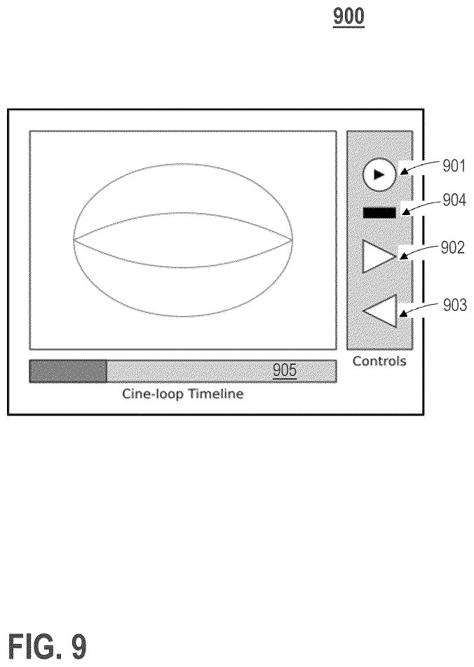

FIG. 9 shows a screenshot 900 displaying a cine-loop during a review process on an ultrasound machine in accordance with aspects of the present disclosure. As shown in FIG. 9, the controls for the reviewing process provide one or more control buttons for performing one or more of: a key control 901 for starting of the playing of the recorded cine-loop, a key control 902 for forwarding the review, a key control 903 for rewinding to a previous time on the timeline, a key control 904 for stopping the review, and a key control 905 for selecting to view the cine-loop at a specific timeline of the recorded cine-loop. As described above, the trackball 380 may be used to review the quality of the recorded images in a timely manner such that further adjustments may be made prior to further scanning.

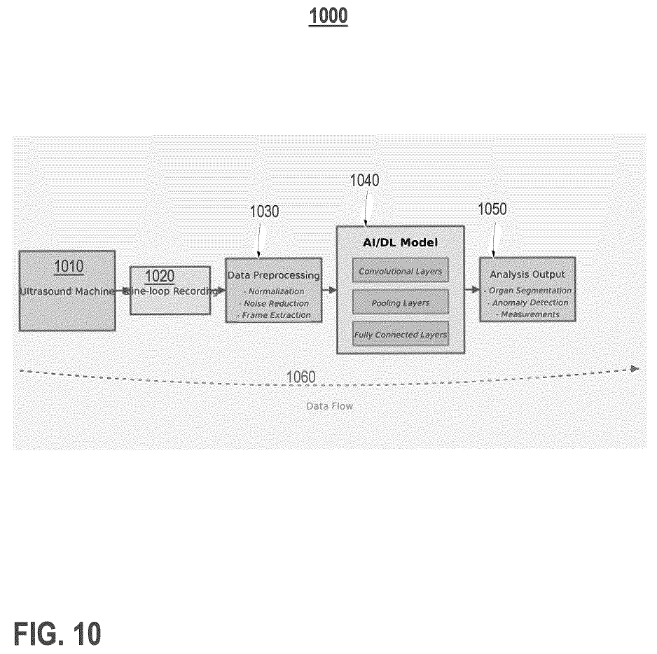

FIG. 10 illustrates a high-level block diagram 1000 showing the feeding of recorded cine-loops into an AI/DL algorithm based analyzer in accordance with aspects of the present disclosure. The block diagram 1000 includes: a block 1010 for performing the scanning via the ultrasound machine, a block 1020 for performing the cine-loop recording, a block 1030 for data preprocessing, such as normalization, noise reduction and frame extraction, a block 1040 for feeding the preprocessed cine-loop recording to the AI/DL based algorithm, and a block 1050 for outputting the results of the analysis. The direction of the dataflow is shown in 1060.

In one aspect, the block 1040 includes convolutional layers 1041 that perform feature extractions for applying linear operations to inputs by multiplying a set of weights with the input, pooling layers 1042 used to reduce the number of parameters and computation, and fully connected layers 1043 for connecting every input to every possible output. In one aspect, the block 1050 is used for organ segmentation, anatomy detection, and for performing measurements.

For example, the AI/DL algorithm of the present disclosure may include a custom model implementing an end-to-end deep-learning algorithm that directly recognizes specific follicles, eliminating the need for post-processing-thereby reducing errors from merging of data. The input to the model may consist of ultrasound scans from transvaginal ultrasound examinations.

In one aspect, the scans are recorded prior to any manual measurements, with sonographers unaware of which images would be used for model development and/or fine-tuning. The model does not require any manual preparation of the input data, such as indications as to the location of the ovary. Instead, the model operates directly on the scans recorded by the ultrasound operator.

The architecture may employ a neural network for image segmentation, such as the U-Net backbone, which contains fully convolutional neural network layers and residual connections. The U-Net backbone is widely used in medical computer vision technologies. This backbone is coupled with modules for the classification, which estimates the likelihood of a region containing a follicle, and indicates the bounding box of the region. These modules for the classification may be based on the You-Only-Look-Once (YOLO) architecture and its later improvements. In order to allow the model to identify 3D outlines of each follicle individually, the present disclosure extends the YOLO architecture with an instance segmentation module. The instance segmentation module extracts specific portions of feature maps corresponding to predicted bounding boxes and generates segmentation masks using a convolution layer. The model produces complete follicle outlines across all frames where each follicle appears, enabling identification of individual follicles. Then, for each follicle, the size measurement may be determined, and the longest diameter on the frame may be identified thereby showing the largest cross-section of each follicle. The measurement techniques are consistent with standard clinical practices.

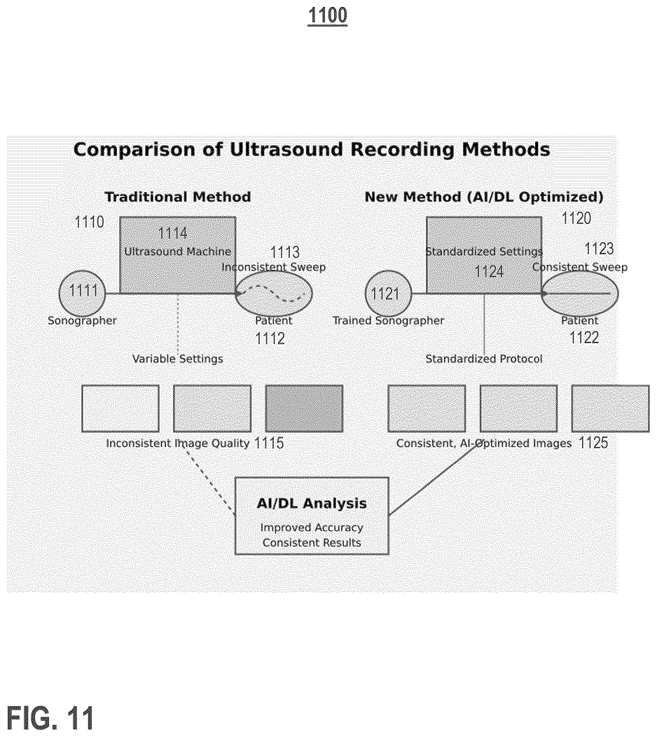

FIG. 11 illustrates a side-by-side comparison 1100 of traditional ultrasound recording techniques 1110 with the ultrasound recording method 1120 in accordance with aspects of the present disclosure. The traditional ultrasound recording technique 1110 includes a sonographer 1111 applying a probe on patient 1112 and performing an inconsistent sweep 1113 and the ultrasound machine 1114 recording images of inconsistent quality 1115 using variable settings of the ultrasound machine. The ultrasound recording technique 1120 of the present disclosure includes a trained sonographer 1121 applying a probe on patient 1122 and performing a consistent sweep 1123 and the ultrasound machine 1124 recording images of consistent quality 1125 using standardized protocols. The images of consistent quality 1125 are AI optimized and are fed to the AI/DL algorithm based analyzer, e.g., as shown in FIG. 10.

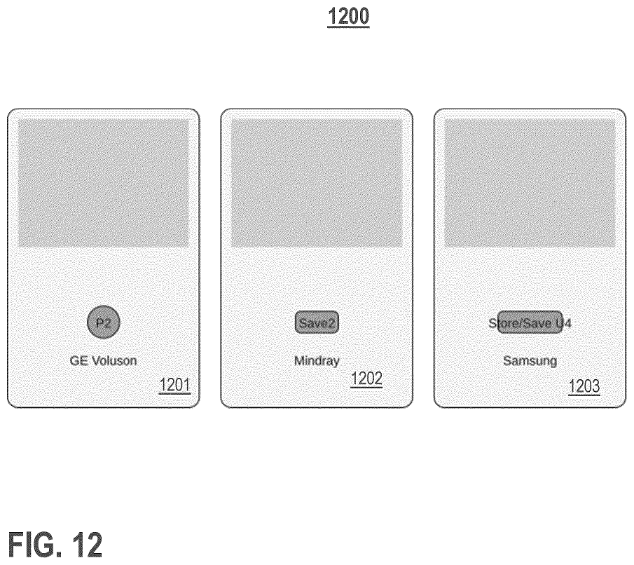

FIG. 12 illustrates high-level block diagrams 1200 of exemplary ultrasound machines on which the method of present disclosure is implemented in accordance with aspects of the present disclosure. The method 200 of the present disclosure may be implemented on any ultrasound machine. For example, the method 200 may be implemented on a GE Voluson ultrasound machine 1201, Mindray ultrasound machine 1202, or a Samsung ultrasound machine 1203, or the like.

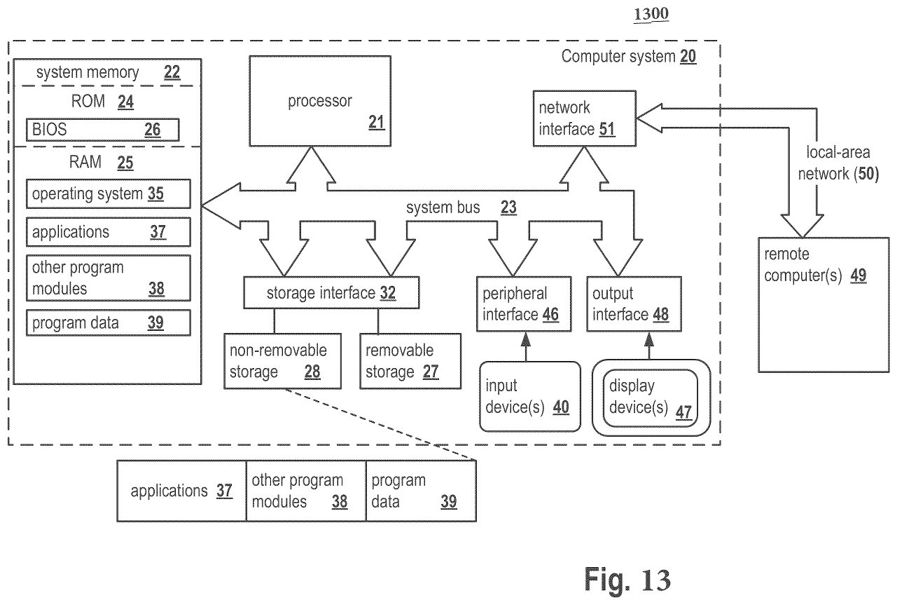

FIG. 13 is a block diagram 1300 illustrating various components of an example computer system 20 via which aspects of the present disclosure may be implemented. That is method 200 may be implemented on the computer system 20.

The computer system 20 may, for example, be or include a computing system of the user device, or may comprise a separate computing device communicatively coupled to the user device, etc. In addition, the computer system 20 may be in the form of multiple computing devices, or in the form of a single computing device, including, for example, a mobile computing device, a cellular telephone, a smart phone, a desktop computer, a notebook computer, a laptop computer, a tablet computer, a server, a mainframe, an embedded device, and other forms of computing devices.

As shown in FIG. 13, the computer system 20 may include one or more central processing units (CPUs) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I²C, and other suitable interconnects. The central processing unit 21 (also referred to as a processor) may include a single or multiple sets of processors having single or multiple cores. The processor 21 may execute one or more computer-executable lines of code implementing techniques in accordance with aspects of the present disclosure. The system memory 22 may be or include any memory for storing data used herein and/or computer programs that are executable via the processor 21. The system memory 22 may include volatile memory, such as a random access memory (RAM) 25 and non-volatile memory, such as a read only memory (ROM) 24, flash memory, etc., or any combination thereof. The basic input/output system (BIOS) 26 may store the basic procedures for transfer of information among elements of the computer system 20, such as those at the time of loading the operating system with the use of the ROM 24.

The computer system 20 may include one or more storage devices, such as one or more removable storage devices 27, one or more non-removable storage devices 28, or a combination thereof. The one or more removable storage devices 27 and non-removable storage devices 28 may be coupled to the system bus 23 via a storage interface 32. In an aspect, the storage devices and the corresponding computer-readable storage media may be or include power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory, such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology, such as in solid state drives (SSDs) or flash drives; magnetic cassettes, magnetic tape, and magnetic disk storage, such as in hard disk drives or floppy disks; optical storage, such as in compact disks (CD-ROM) or digital versatile disks (DVDs); and any other medium that may be used to store the desired data and that may be accessed via the computer system 20.

The system memory 22, removable storage devices 27, and/or non-removable storage devices 28 of the computer system 20 may be used to store an operating system 35, additional program applications 37, other program modules 38, and/or program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47, such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

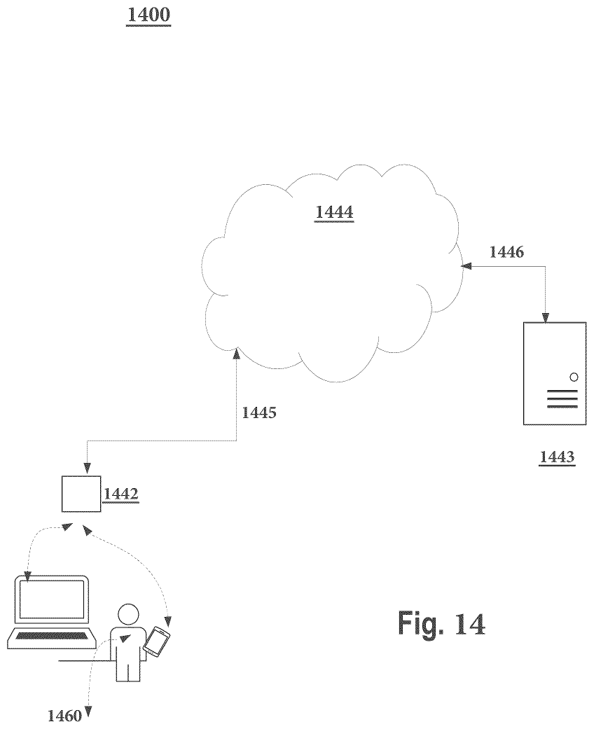

The computer system 20 may operate in a network environment as shown in FIG. 14, using a network connection to one or more remote computers 49. The remote computer (or computers) 49 may be or include local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks, such as a local-area computer network (LAN) 50, a wide-area computer network (WAN), an intranet, and the Internet. Examples of the network interface 51 may include an Ethernet interface, a Frame Relay interface, SONET interface, and wireless interfaces.

FIG. 14 is a block diagram of various example system components, usable in accordance with aspects of the present disclosure. FIG. 14 shows a communication system 1400 usable in accordance with aspects of the present disclosure. The communication system 1400 includes one or more accessors 1460 (also referred to interchangeably herein as one or more “users”) and one or more terminals 1442. In one aspect, data for use in accordance with aspects of the present disclosure may, for example, be input and/or accessed by accessors 1460 via terminals 1442, such as personal computers (PCs), minicomputers, mainframe computers, microcomputers, telephonic devices, or wireless devices, such as personal digital assistants (“PDAs”), smart phones, or other hand-held wireless devices coupled to a server 1443, such as a PC, minicomputer, mainframe computer, microcomputer, or other device having a processor and a repository for data and/or connection to a repository for data, via, for example, a network 1444, such as the Internet or an intranet, and couplings 1445, 1446. In one aspect, various features of the method may be performed in accordance with a command received from another device via a coupling 1445, 1446. The couplings 1445, 1446 may include, for example, wired, wireless, or fiberoptic links. In another variation, various features of the method and system in accordance with aspects of the present disclosure may operate in a stand-alone environment, such as on a single terminal. In one aspect, the server 1443 may be a remote computer 49, as shown in FIG. 13, or a local server.

Aspects of the present disclosure may be or include a system, a method, and/or a computer program product. The computer program product may include a computer readable storage medium (or media) having computer readable program instructions thereon for causing a processor to carry out aspects of the present disclosure.

The computer readable storage medium may be or include a tangible device that may retain and store program code in the form of instructions or data structures that may be accessed via a processor of a computing device, such as the computing system 20. The computer readable storage medium may be or include an electronic storage device, a magnetic storage device, an optical storage device, an electromagnetic storage device, a semiconductor storage device, or any suitable combination thereof. By way of example, such computer-readable storage medium may comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device, such as punch-cards or raised structures in a groove having instructions recorded thereon. As used herein, a computer readable storage medium is not to be construed as being or only being transitory signals per se, such as radio waves or other freely propagating electromagnetic waves, electromagnetic waves propagating through a waveguide or transmission media, or electrical signals transmitted through a wire.

Computer readable program instructions described herein may be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations in accordance with aspects of the present disclosure may be or include assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be coupled to the user's computer via any suitable type of network, including a LAN or WAN, or the connection may be made to an external computer (for example, through the Internet). In some aspects, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform various functions in accordance with aspects of the present disclosure.

In various aspects, the systems and methods described in the present disclosure may be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system (such as the one described in greater detail in FIG. 13, above). Accordingly, each module may be realized in a variety of suitable configurations, and should not be limited to any particular implementation shown or described as an example herein.

In the interest of clarity, not all of the routine features of the aspects are disclosed herein. It will be appreciated that in the development of any actual implementation of features in accordance with aspects of the present disclosure, numerous implementation-specific decisions may be made in order to achieve the developer's specific goals, and these specific goals may vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of various features in accordance with aspects of the present specification are to be interpreted by one of ordinary skill in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it will be apparent to those skilled in the art having the benefit of this disclosure that many more modifications than mentioned above are possible without departing from the innovative concepts disclosed herein.

What is claimed is:

1. A computer-implemented method for recording ultrasound videos designed to facilitate analysis by AI and/or DL algorithms comprising:
selecting an appropriate preset on an ultrasound device for an application at least in part based on a target organ;
positioning a probe of the ultrasound machine thereby positioning an image of the target organ at a center of a screen of a display device;
performing adjustments of scanning depths until an entirety of the target organ is visualized with minimal surrounding structures;
performing image enhancements, the performing of the image enhancements including one or more of: enabling Harmonic Imaging (HI) or Full Spectral Imaging (FSI), when the HI or FSI enhancement modes are available on the ultrasound machine, adjusting gains and/or contrasts when adjustments to gains or contrasts are needed;
selecting a scanning plane for a complete sweep from one end of the target organ to an opposite end of the target organ;
performing an initial positioning of the probe;
initiating the cine-loop recording;
scanning the target organ from the one end of the target organ to the opposite end of the target organ while recording the cine-loop, the scanning being performed with a steady, uniform motion, maintaining a constant scanning plane; and
ending the cine-loop recording when the scanning is completed, and storing the recorded cine-loop in a memory of the ultrasound machine.

2. The computer-implemented method of claim 1, further comprising:
performing a review of the recorded cine-loop for quality, and determining whether the recorded cine-loop satisfied criteria for subsequent analysis.

3. The computer-implemented method of claim 2, further comprising:
providing an operator of the ultrasound machine an opportunity to adapt techniques used during the scanning for future scans.

4. The computer-implemented method of claim 1, wherein the target organ is an ovary or a uterus and the method is used to conduct gynecological studies, the gynecological studies comprising at least simulations of in vitro fertilization (IVF).

5. The computer-implemented method of claim 4, wherein the target organ is the ovary and the method is used for counting and measuring follicles in the gynecological studies, the gynecological studies comprising at least the simulations of the IVF.

6. The computer-implemented method of claim 4, wherein the target organ is the uterus and the method is used for measuring an endometrium thickness in the gynecological studies, the gynecological studies comprising at least the simulations of the IVF.

7. The computer-implemented method of claim 1, wherein the cine-loop recording is optimized for use with a specific ultrasound machine, the specific ultrasound machine being at least an ultrasound machine selected from: a GE Voluson ultrasound machine, a Siemens ultrasound machine, a Samsung ultrasound machine, a Philips ultrasound machine, a Canon Aplio ultrasound machine, a Mindray ultrasound machine, a Fujifilm ultrasound machine, a BK Medical ultrasound machine, and a Hitachi ultrasound machine.

8. A system for recording ultrasound videos of a target organ, comprising:
a computing with memory, the computer including at least one processor;
a display device communicatively coupled to the computing device;
a beamforming equipment communicatively coupled to the display device and the computer;
an ultrasound machine for performing scans comprising a transducer coupled to the beamforming equipment; and
a navigation support program stored on the memory of the computer, the navigation support program including software code, the code when executed by the processor of the computer performing:
capturing of images of an organ withing a body using the ultrasound machine; and
processing information gathered during the capturing of the images to create a repeating video sequence optimized for automated analysis by an AI and/or DL based algorithm.

9. The system of claim 8, wherein the target organ is an ovary or a uterus and the system is used to conduct gynecological studies, the gynecological studies comprising at least simulations of in vitro fertilization (IVF).

10. The system of claim 9, wherein the target organ is the ovary and the system is used for counting and measuring follicles in the gynecological studies, the gynecological studies comprising at least the simulations of the IVF.

11. The system of claim 9, wherein the target organ is the uterus and the system is used for measuring an endometrium thickness in the gynecological studies, the gynecological studies comprising at least the simulations of the IVF.

12. The system of claim 8, wherein the cine-loop recording is optimized for use with a specific ultrasound machine, the specific ultrasound machine being at least an ultrasound machine selected from: a GE Voluson ultrasound machine, a Siemens ultrasound machine, a Samsung ultrasound machine, a Philips ultrasound machine, a Canon Aplio ultrasound machine, a Mindray ultrasound machine, a Fujifilm ultrasound machine, a BK Medical ultrasound machine, and a Hitachi ultrasound machine.

13. A computer program product for ultrasound navigation during recording of videos of a target organ, the computer program product comprising instructions stored on a computer-readable medium, when executing by a computing device, the instructions enabling the computer to perform:
receiving ultrasound data of the target organ in raw form an ultrasound machine when the ultrasound machine performing scanning;
processing the received ultrasound data to generate a cine-loop optimized for automated analysis by an AI and/or DL based algorithm in accordance with the method of claim 1; and
storing the generated cine-loop in a memory of the computing device.

14. The computer program product of claim 13, wherein the target organ is an ovary or a uterus and the computer program product is used to conduct gynecological studies, the gynecological studies comprising at least simulations of in vitro fertilization (IVF).

15. The computer program product of claim 14, wherein the target organ is the ovary and the computer program product is used for counting and measuring follicles in the gynecological studies, the gynecological studies comprising at least the simulations of the IVF.

16. The computer program product of claim 14, wherein the target organ is the uterus and the computer program product is used for measuring an endometrium thickness in the gynecological studies, the gynecological studies comprising at least the simulations of the IVF.

17. The computer program product of claim 13, wherein the cine-loop recording is optimized for use with a specific ultrasound machine, the specific ultrasound machine being at least an ultrasound machine selected from: a GE Voluson ultrasound machine, a Siemens ultrasound machine, a Samsung ultrasound machine, a Philips ultrasound machine, a Canon Aplio ultrasound machine, a Mindray ultrasound machine, a Fujifilm ultrasound machine, a BK Medical ultrasound machine, and a Hitachi ultrasound machine.

18. A computer-implemented system is provided for recording ultrasound videos to facilitate automated analysis by AI and DL algorithms, the system comprising:
at least one memory; and
at least one hardware processor coupled with the at least one memory and configured, individually or in combination, to:
select an appropriate preset on an ultrasound device for an application at least in part based on a target organ;
position a probe of the ultrasound machine thereby positioning an image of the target organ at a center of a screen of a display device;
perform adjustments of scanning depths until an entirety of the target organ is visualized with minimal surrounding structures;
perform image enhancements, the performing of the image enhancements including one or more of: enabling Harmonic Imaging (HI) or Full Spectral Imaging (FSI), when the HI or FSI enhancement modes are available on the ultrasound machine, adjusting gains and/or contrasts when adjustments to gains or contrasts are needed;
select a scanning plane for a complete sweep from one end of the target organ to an opposite end of the target organ;
perform an initial positioning of the probe;
initiate the cine-loop recording;
scan the target organ from the one end of the target organ to the opposite end of the target organ while recording the cine-loop, the scanning being performed with a steady, uniform motion, maintaining a constant scanning plane;
end the cine-loop recording when the scanning is completed, and store the recorded cine-loop in a memory of the ultrasound machine;
perform a review of the recorded cine-loop for quality, and determine whether the recorded cine-loop satisfied criteria for subsequent analysis; and
provide an operator of the ultrasound machine an opportunity to adapt techniques used during the scanning for future scans.

19. A non-transitory computer-readable medium is provided storing a set of instructions thereon for recording ultrasound videos to facilitate automated analysis by AI and DL algorithms, wherein the set of instructions comprises instructions for:
selecting an appropriate preset on an ultrasound device for an application at least in part based on a target organ;
positioning a probe of the ultrasound machine thereby positioning an image of the target organ at a center of a screen of a display device;
performing adjustments of scanning depths until an entirety of the target organ is visualized with minimal surrounding structures;
performing image enhancements, the performing of the image enhancements including one or more of: enabling Harmonic Imaging (HI) or Full Spectral Imaging (FSI), when the HI or FSI enhancement modes are available on the ultrasound machine, adjusting gains and/or contrasts when adjustments to gains or contrasts are needed;
selecting a scanning plane for a complete sweep from one end of the target organ to an opposite end of the target organ;
performing an initial positioning of the probe;
initiating the cine-loop recording;
scanning the target organ from the one end of the target organ to the opposite end of the target organ while recording the cine-loop, the scanning being performed with a steady, uniform motion, maintaining a constant scanning plane;
ending the cine-loop recording when the scanning is completed and storing the recorded cine-loop in a memory of the ultrasound machine;
performing a review of the recorded cine-loop for quality, and determining whether the recorded cine-loop satisfied criteria for subsequent analysis; and
providing an operator of the ultrasound machine an opportunity to adapt techniques used during the scanning for future scans.
